(19) United States
(12) Patent Application Publication
(10) Pub. No.: US 2026/0328763 A1
(43) Pub. Date: Oct. 22, 2026

(54) MULTIPLEX LATERAL FLOW IMMUNOASSAY DEVICE, METHOD, AND SYSTEM FOR DETECTION OF ANALYTES IN WHOLE BLOOD

(71) Applicant: QikDx Holdings Inc., Surrey (CA)

(72) Inventors: Razieh Salahandish, Toronto (CA); Fatemeh Rahimi, North York (CA); Elnaz Haghani, North York (CA); Ashissh Raichura, Vancouver (CA)

(21) Appl. No.: 19/646,447

(22) Filed: Apr. 13, 2026

Related U.S. Application Data
(60) Provisional application No. 63/789,710, filed on Apr. 16, 2025.

(51) Int. Cl. G01N 33/543; G01N 21/78

(52) U.S. Cl. CPC G01N 33/54388; G01N 21/78; G01N 33/54346

(57) ABSTRACT

A multiplex lateral flow immunoassay device, method, and system for detecting a plurality of analytes in a whole blood sample are provided. The device includes a capillary-driven fluidic network defined by a substrate layer, one or more bonding layers forming microfluidic channels, and a porous membrane. A blood-separation element at or upstream of inlet regions retains cellular blood components while permitting plasma to pass into the fluidic network. A plurality of spatially separated detection channels each include a conjugate zone with detection reagents and a sensing zone coated with a capture agent. The device performs a competitive immunoassay in each channel such that signal intensity at each sensing zone varies inversely with analyte concentration. A mobile application captures an image of the sensing zones, and a processor determines analyte concentrations based on calibration data. The platform enables multiplex detection from a finger-prick sample without external sample preparation.

### BACKGROUND

Point-of-care diagnostics require accurate detection and quantification of biomarkers at or near the site of patient care. Clinicians, researchers, and patients have long sought methods to measure multiple biomarkers simultaneously from minimally invasive samples, without relying on centralized laboratory infrastructure.

Laboratory-based immunoanalytical platforms for steroid hormone quantification, including cortisol, testosterone, and progesterone, typically cost hundreds of thousands of dollars and require dedicated space, maintenance contracts, and trained personnel. For patients needing progesterone monitoring during fertility treatment or individuals tracking hormones for athletic performance, these barriers make centralized testing burdensome and costly, with turnaround times of 24-72 hours that are too slow for time-sensitive decisions.

Chromatographic mass spectrometry offers high specificity and can distinguish structurally similar steroids in a single analytical run. However, this instrumentation is costly, requires trained operators and controlled laboratory environments, and involves complex sample preparation workflows. Turnaround times span hours to days, making these methods impractical for point-of-care or home-based monitoring.

Lateral flow immunoassay (LFIA) technology is widely used for point-of-care diagnostics due to its low cost, stability, and ease of use. However, adapting LFIA to small molecules like steroid hormones is challenging. Small molecules present only a single epitope and cannot be detected using sandwich immunoassays, which require two binding sites. Instead, competitive immunoassays are required, where signal intensity is inversely correlated with analyte concentration, complicating quantitative readout.

Most lateral flow devices detect only a single analyte per strip. True multiplexing requires spatially separated detection channels, each with its own reagent chemistry, configured to minimize cross-reactivity and maintain consistent capillary transport. These design constraints become more demanding as the number of target analytes increases. Working with whole blood adds further constraints. Red blood cells and hemoglobin can interfere with colorimetric detection and impede flow uniformity. Typically, whole blood must be centrifuged or processed before application to a lateral flow strip, adding steps and equipment that reduce point-of-care practicality.

Conventional visual interpretation of lateral flow results is subjective and qualitative. Smartphone-based imaging can improve objectivity and enable quantitative results, but reliable quantification requires accounting for variable lighting, camera differences, and inconsistent user technique. Without on-device calibration and robust image processing, mobile-based readouts remain limited in accuracy and reproducibility.

Accordingly, a need exists for an integrated device and system that can accept unprocessed whole blood, separate plasma on-device, execute parallel competitive immunoassays with minimal cross-reactivity, and deliver calibrated, quantitative results through mobile imaging, all without requiring centralized laboratory infrastructure, skilled operators, or external sample preparation.

### SUMMARY

Examples herein relate to a multiplex lateral flow immunoassay device for the detection of a plurality of analytes in a whole blood sample. An example device includes a capillary-driven fluidic network defined by at least a substrate layer, one or more bonding layers patterned to form a plurality of microfluidic channels, and a porous membrane disposed within the channels, the fluidic network extending from a plurality of inlet regions through the porous membrane. In the example device, a blood-separation element is disposed at or upstream of each inlet region, the blood-separation element configured to selectively retain cellular blood components while permitting plasma to pass into the fluidic network.

In an example, the device also includes a plurality of spatially separated detection channels formed within the fluidic network, each detection channel including an inlet region, a conjugate zone containing detection reagents conjugated to antibodies specific to a respective target analyte, and a sensing zone on the porous membrane coated with a capture agent corresponding to the respective target analyte. The device is configured to perform a competitive immunoassay in each detection channel such that signal intensity at each sensing zone is inversely correlated with the concentration of the respective target analyte in the sample.

In some examples, the porous membrane includes nitrocellulose, and one or more bonding layers include a pressure-sensitive adhesive (PSA). The nitrocellulose membrane serves as the primary sensing substrate, supporting capillary-driven fluid transport and hosting the immobilized capture agents at each sensing zone. For example, the PSA bonding layers are patterned by laser cutting to define the microfluidic channel geometry, confining fluid flow to the defined pathways and providing a hydrophobic barrier that prevents cross-channel contamination. In some examples, each detection channel is fluidically isolated from the other detection channels, ensuring that the competitive immunoassay in each channel proceeds independently without cross-reactivity between target analytes.

In some examples, the detection reagents include gold nanoparticles conjugated to monoclonal antibodies specific to the respective target analyte. The gold nanoparticles accumulate at each sensing zone in inverse proportion to the concentration of the corresponding free analyte in the plasma, generating a colorimetric signal that is directly readable by an optical measurement system. For example, at zero analyte concentration, maximum gold-nanoparticle accumulation produces a dark red colorimetric signal at the sensing zone, whereas at elevated analyte concentrations, reduced gold-nanoparticle capture yields an attenuated or absent signal. In some examples, the gold nanoparticles have a mean diameter between about 15 nm and about 30 nm, a size range that provides strong surface plasmon resonance absorption suitable for colorimetric detection. In some examples, the blood-separation element includes a plasma separation membrane configured to retain red blood cells while permitting plasma to pass, eliminating the need for pre-processing or centrifugation of the whole blood sample prior to introduction into the fluidic network.

In an example, the device further includes a calibration region including fiducial markers and color reference standards to facilitate optical readout. The fiducial markers enable automatic correction for perspective distortion and misalignment during image acquisition, while the color reference standards enable normalization of colorimetric signals across variable illumination conditions and different imaging devices. For example, a smartphone application captures an image of the device and uses the fiducial markers to geometrically register the sensing zones before applying color normalization based on the integrated reference standards.

Examples herein also relate to a method for detecting a plurality of analytes in a whole blood sample using a multiplex lateral flow immunoassay device. The method includes applying the whole blood sample to a plurality of inlet regions of the device, the device having a blood-separation element, a capillary-driven fluidic network including a porous membrane, and a plurality of spatially separated detection channels. Cellular components are separated from the sample with the blood-separation element to yield plasma that enters the fluidic network. The plasma and detection reagents in each detection channel are transported, by capillary action, through the porous membrane to a respective sensing zone corresponding to a respective target analyte. Competitive binding is induced such that a signal intensity at each sensing zone varies inversely with concentration of the respective target analyte. An optical measurement of the sensing zones is acquired and concentrations of the plurality of analytes are determined based on stored calibration data.

In some examples, acquiring the optical measurement includes capturing an image with a camera of a mobile device and analyzing the image with software executing on the mobile device. For example, determining concentrations can include converting image data to a perceptually uniform color space and calculating a color-difference metric relative to a reference standard. In an example, the software converts pixel values from each sensing zone into a color space such as the International Commission on Illumination color space (CIELAB) and calculates a total color-difference metric, designated ΔE, for each sensing zone relative to a blank baseline condition, mapping the resulting ΔE values to analyte concentrations using pre-established calibration models.

In some examples, the method further includes normalizing the optical measurement using fiducial markers and color reference standards on the device, compensating for variations in ambient illumination and camera spectral response across different mobile devices and environments. In some examples, the whole blood sample is a finger-prick sample, and the separating step is performed by a plasma separation membrane that retains red blood cells, enabling point-of-care use without laboratory infrastructure or centrifugation equipment.

Examples herein further relate to a system for detecting a plurality of analytes in a whole blood sample. The system includes a multiplex lateral flow immunoassay device having a blood-separation element, a capillary-driven fluidic network including a porous membrane, and a plurality of spatially separated detection channels formed within the fluidic network, each with a respective sensing zone, wherein the device is configured to perform a competitive immunoassay in each detection channel such that signal intensity at each sensing zone varies inversely with concentration of a respective target analyte. The system further includes a mobile application. The application can perform image-based analysis of the captured image, including quantification of signal intensity at each sensing zone relative to a reference. The application then determines concentrations of the plurality of analytes based on calibration data and generates an output indicative of the determined concentrations. In some examples, the camera, memory, and processor are implemented in a single mobile device, enabling fully self-contained point-of-care operation without external instrumentation.

Both the foregoing general description and the following description are exemplary and explanatory only and are not restrictive of the examples, as claimed.

### BRIEF DESCRIPTION OF THE DRAWINGS

FIG. 1 is a flowchart of an example method for detecting a plurality of analytes in a whole blood sample according to one or more embodiments herein.

FIG. 2 is a system diagram of an example system for detecting a plurality of analytes in a whole blood sample according to one or more embodiments herein.

FIG. 3A is a schematic of an example assembled multiplex lateral flow immunoassay device according to one or more embodiments herein.

FIG. 3B is an exploded view of the example multiplex lateral flow immunoassay device of FIG. 3A according to one or more embodiments herein.

FIG. 4 is a depiction of time-resolved capillary flow through the example device according to one or more embodiments herein.

FIGS. 5A-5C are depictions of device responses to increasing analyte concentrations according to one or more embodiments herein.

FIGS. 6A-6B are depictions of mixed-analyte device responses and corresponding calibration curves according to one or more embodiments herein.

FIG. 7 is a depiction of selectivity results under single-analyte and binary-mixture conditions according to one or more embodiments herein.

FIG. 8 is a depiction of whole-blood operation and mobile device readout according to one or more embodiments herein.

FIG. 9 is a schematic of an alternative multiplex test strip embodiment according to one or more embodiments herein.

### DETAILED DESCRIPTION

Reference will now be made in detail to the present examples, including examples illustrated in the accompanying drawings.

The present disclosure relates to multiplex lateral flow immunoassay devices, methods, and systems configured to detect a plurality of analytes in a whole blood sample. In some examples, a device includes a capillary-driven fluidic network defined by a substrate layer, one or more bonding layers patterned to form microfluidic channels, and a porous membrane disposed within the channels. The device can further include a blood-separation element disposed at or upstream of one or more inlet regions and a plurality of spatially separated detection channels, each having a respective sensing zone. The device is configured to perform a competitive immunoassay in each detection channel such that signal intensity at each sensing zone varies inversely with the concentration of a respective target analyte.

In some examples, each detection channel comprises an inlet region, a conjugate zone including detection reagents conjugated to antibodies specific to a respective target analyte, and a sensing zone on the porous membrane coated with a capture agent corresponding to the respective target analyte. In operation, whole blood applied at the inlet is separated to yield plasma that enters the capillary-driven fluidic network. Plasma and detection reagents are transported by capillary action to the sensing zones, where competitive binding occurs and produces a measurable signal that decreases with increasing analyte concentration.

In further examples, the device is read optically. A camera, which can be a camera of a mobile device, captures an image of the sensing zones. A processor, which can be implemented on the mobile device or another computing device, executes instructions stored in a memory to process the image and determine analyte concentrations. In some embodiments, the device includes a calibration region comprising fiducial markers and color reference standards, and the processor normalizes the image using the fiducial markers and color reference standards. In some examples, determining concentrations includes calculating a color-difference metric for each sensing zone relative to a reference and mapping the metric to concentration using calibration data.

In other examples, the device architecture is configured to promote fluidic isolation among detection channels while maintaining uniform capillary transport. The blood-separation element is configured to selectively retain cellular blood components while permitting plasma to pass into the fluidic network, enabling direct use with finger-prick whole blood without external sample preparation. The porous membrane can comprise nitrocellulose, and one or more bonding layers can comprise a pressure-sensitive adhesive, although other suitable materials can be used.

Although cortisol, testosterone, and progesterone are described herein as representative target analytes, the platform is applicable to other low-molecular-weight analytes detectable by competitive immunoassay, including vitamins, electrolytes, metabolites, therapeutic drugs, and environmental small molecules. The number of detection channels, conjugate pads, and sensing zones can be adjusted to accommodate different analyte panels without departing from the architecture described herein.

The disclosure also provides systems and methods of use. An example method includes applying whole blood to a plurality of inlet regions of a multiplex device, separating cellular components to yield plasma that enters the fluidic network, transporting plasma and detection reagents by capillary action to respective sensing zones, performing competitive binding such that signal intensity varies inversely with analyte concentration, and acquiring an optical measurement and determining concentrations based on calibration data. An example system includes a multiplex lateral flow immunoassay device as described herein, and a mobile application. The application can capture an image of all sensing zones together and perform image-based analysis of the captured image to determine concentrations of a plurality of analytes, and generate an output indicating the concentrations. For example, a camera configured to capture an image of the sensing zones, and a computing device comprising a processor and a memory storing instructions that, when executed by the processor, cause the computing device to process the image, determine concentrations of a plurality of analytes, and generate an output indicating the concentrations.

In various examples, detection reagents can include gold nanoparticles conjugated to monoclonal antibodies, and the blood-separation element can include a plasma separation membrane configured to retain red blood cells while permitting plasma to pass. At least some of the examples described are suitable for point-of-care or at-home use and can reduce reliance on centralized laboratory testing by enabling multiplex detection of multiple analytes from a single whole blood sample.

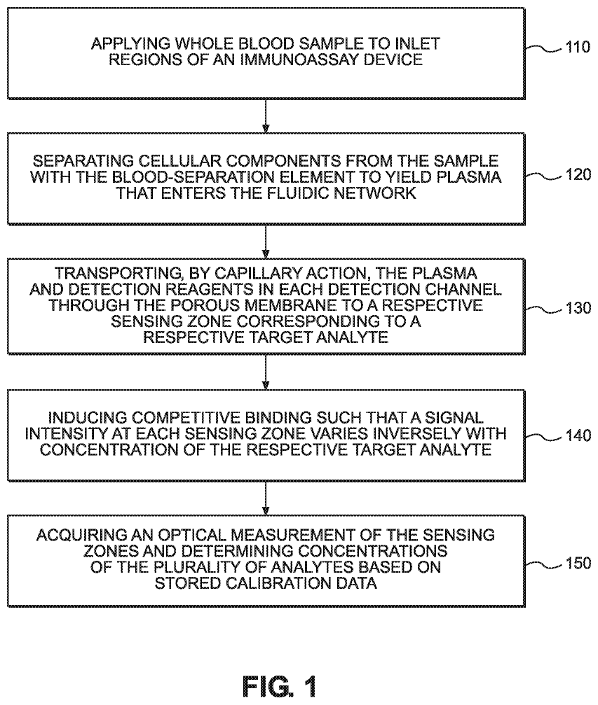

FIG. 1 depicts a flowchart of an example method 100 for detecting a plurality of analytes in a whole blood sample using a multiplex lateral flow immunoassay device according to examples herein. The method can be performed, in an example, using a multiplex lateral flow immunoassay device as described below. The method can represent a sequence of operations through which an unprocessed whole blood sample is transformed into quantified analyte concentrations, but in some examples, the stages of the method can be performed in different orders, where relevant.

Stage 110 can include applying a whole blood sample to a plurality of inlet regions of the device. For example, the whole blood sample can be obtained by finger-prick and deposited directly onto the inlet regions without external preprocessing such as centrifugation. In some examples, the plurality of inlet regions correspond to respective detection channels of the device, such that each inlet region receives a portion of the applied sample. The applied sample volume can be sufficient to initiate capillary-driven flow through the device.

Stage 120 can include separating cellular components from the sample with a blood-separation element to yield plasma that enters a capillary-based fluidic network, including a porous membrane. In this example, “capillary-based” means the same as “capillary-driven,” indicating that no separate pumping mechanism is required. In some examples, the blood-separation element is disposed at or upstream of each inlet region and is configured to selectively retain cellular blood components, such as red blood cells, while permitting plasma to pass into the fluidic network. For example, the blood-separation element can be a plasma separation membrane positioned on top of the inlet regions. This integrated separation eliminates the need for external sample preparation, such as centrifugation or pipetting.

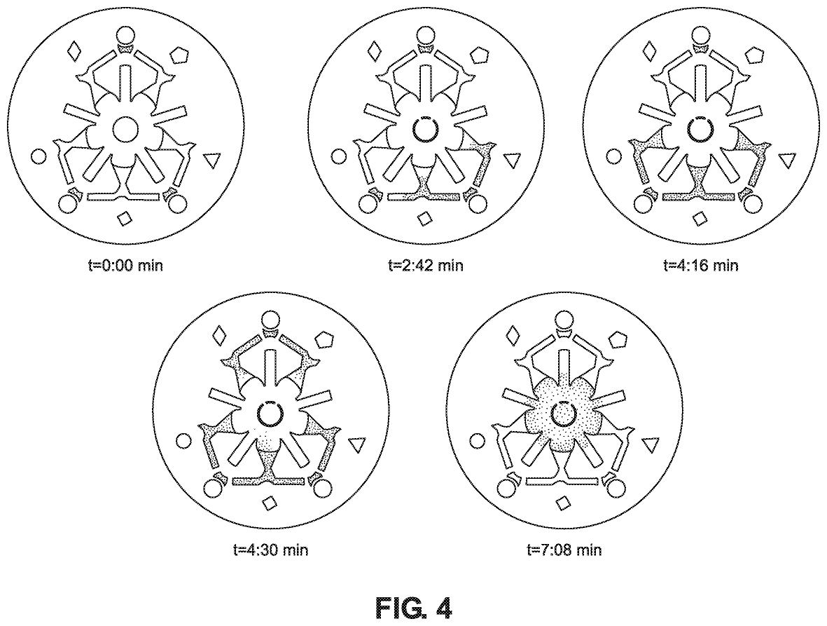

Stage 130 can include transporting, by capillary action, the plasma and detection reagents in each of a plurality of spatially separated detection channels through the porous membrane to a respective sensing zone corresponding to a respective target analyte. In some examples, each detection channel includes a conjugate zone containing detection reagents conjugated to antibodies specific to the respective target analyte, and the detection reagents are rehydrated upon contact with the plasma. The capillary-driven transport proceeds without external pumps or power. For example, as described below with respect to FIG. 4, transport can proceed uniformly across all detection channels and reach a downstream region within minutes.

Stage 140 can include inducing competitive binding in each detection channel such that a signal intensity at each sensing zone varies inversely with the concentration of the respective target analyte. In some examples, each sensing zone on the porous membrane is coated with a capture agent corresponding to the respective target analyte. In the absence of free analyte, the detection reagents bind to the capture agent, producing a strong signal. As analyte concentration increases, free analyte molecules compete for binding sites on the detection reagents, reducing the amount of detection reagent available for capture at the sensing zone and resulting in a proportional decrease in signal intensity.

Stage 150 can include acquiring an optical measurement of the sensing zones and determining concentrations of the plurality of analytes based on stored calibration data. In some examples, a camera of a mobile device captures an image of the sensing zones, and a processor of the mobile device executes instructions stored in a memory to process the image and determine concentrations. For example, the processor can normalize the image using fiducial markers and color reference standards on the device, as described in more detail below. In certain examples, determining concentrations includes converting image data to a perceptually uniform color space and calculating a color-difference metric relative to a reference. The determined concentrations can be presented as an output on a display of the mobile device.

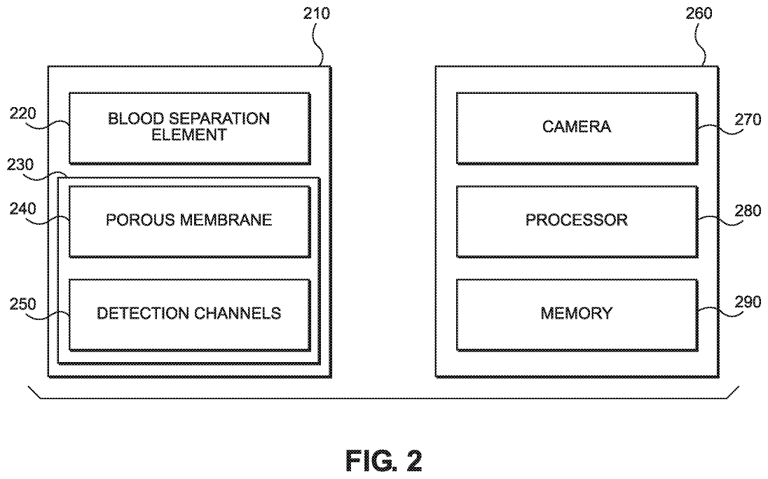

FIG. 2 depicts an example system 200 for detecting a plurality of analytes in a whole blood sample according to examples herein. The system 200 includes a multiplex lateral flow immunoassay (LFIA) device 210 and a mobile device 260. The mobile device 260 is configured to acquire and process images of the device 210 to produce quantitative analyte concentration outputs.

In an example, the device 210 includes a blood-separation element 220 and a capillary-based fluidic network 230. The fluidic network 230 includes a porous membrane 240 and a plurality of spatially separated detection channels 250, each with a respective sensing zone. The blood-separation element 220 is disposed at or upstream of one or more inlet regions and selectively retains cellular blood components while permitting plasma to pass into the capillary-based fluidic network 230. That plasma then contacts the porous membrane 240, which serves as both a capillary-driven transport medium and a substrate for the sensing zones.

The plurality of spatially separated detection channels 250 are formed within the capillary-based fluidic network 230 and are arranged to reduce cross-talk between channels. Each detection channel includes an inlet region, a conjugate zone with detection reagents specific to a respective target analyte, and a respective sensing zone on the porous membrane 240. For example, in use, the device 210 performs a competitive immunoassay in each detection channel such that signal intensity at each respective sensing zone varies inversely with the concentration of the respective target analyte.

In an example, the mobile device 260 includes a camera 270, a processor 280, and a memory 290. The camera 270 captures an image of the sensing zones of the device 210 and passes that image to the processor 280 for analysis. The memory 290 stores calibration data and executable instructions that, when executed by the processor 280, cause the processor 280 to process the captured image, determine concentrations of a plurality of analytes based on the calibration data, and generate an output indicating those concentrations.

In some examples, processing the image includes normalizing the image using features of the device 210, such as fiducial markers and color reference standards, segmenting regions of interest corresponding to the sensing zones, and calculating a color-based metric relative to a reference standard. The resulting metric is then mapped to concentration values using the calibration data stored in the memory 290. For example, the output can be presented on a display of the mobile device 260, stored locally on the device, or communicated to a remote system for further analysis.

The camera 270 can be provided by the mobile device 260 or by another imaging device. In some examples, at least a portion of the processing described herein can be performed by a remote computing resource, and the processor 280 and memory 290 can represent or include components of such a resource. In certain examples, the system 200 can further include accessories such as a cradle or enclosure to assist with image acquisition, and additional sensors or indicators to confirm device validity or flow completion.

In some examples, the imaging device is a dedicated optical reader rather than a mobile device. The dedicated reader can include a housing configured to receive and position the device 210, a controlled illumination source, and a fixed imaging sensor, providing consistent illumination and geometric alignment across readings without reliance on ambient lighting or handheld image capture.

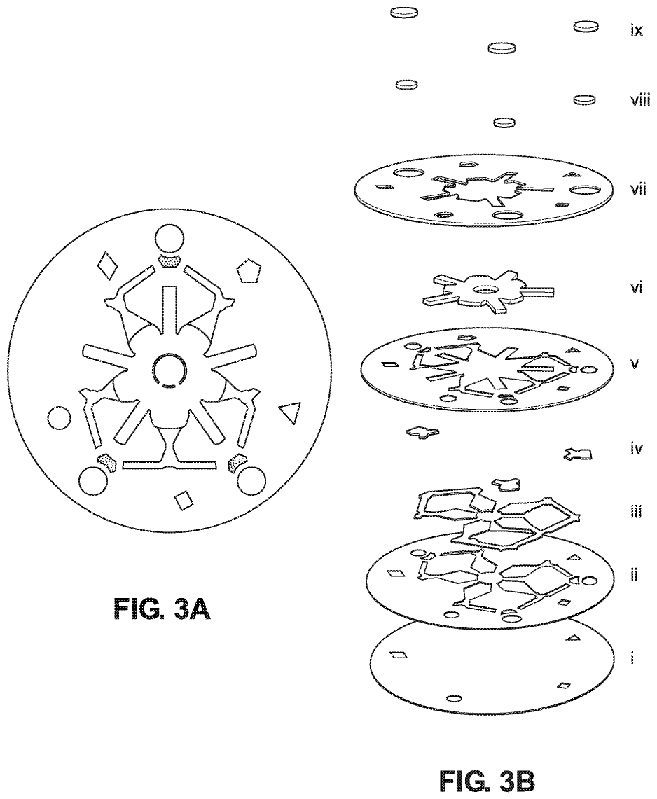

FIGS. 3A and 3B depict an example multiplex lateral flow immunoassay device according to examples herein. FIG. 3A illustrates the device in an assembled configuration, presenting external features used during operation, while FIG. 3B illustrates an exploded view of various layers that, when assembled, form the multiplex lateral flow immunoassay device.

In the assembled view of FIG. 3A, the device presents a plurality of inlet regions configured to receive a whole blood sample. In some examples, a blood-separation element is disposed at or over the inlet regions and is configured to selectively retain cellular blood components while permitting plasma to pass into the device. The device includes a plurality of spatially separated detection channels formed within the body of the device, each detection channel including a respective sensing zone on a porous membrane. In use, the sensing zones can develop a visually detectable signal whose intensity varies inversely with the concentration of a respective target analyte under a competitive immunoassay format. In certain examples, the device further includes a downstream absorbent region to draw fluid by capillary action and a calibration region with fiducial markers and color reference standards to facilitate optical readout.

In the exploded view of FIG. 3B, the device includes components labeled (i) through (ix). Component (i) can include a substrate layer, such as a polyethylene terephthalate (PET) sheet, providing mechanical support and dimensional stability for the device during fabrication and use. Component (ii) can include a patterned pressure-sensitive adhesive (PSA) layer that serves as a structural interface, bonding adjacent layers and defining microfluidic features by way of laser-cut apertures and channels. In some examples, the pattern of component (ii) establishes walls and interfaces for sample routing, conjugate release, and sensing-zone placement.

Component (iii) can include a porous membrane, such as a nitrocellulose (NC) membrane, disposed within openings formed by the patterned PSA to provide a capillary-driven transport medium and a substrate for sensing regions. In some examples, component (iii) includes one or more sensing zones defined on the porous membrane and configured to generate a measurable signal under a competitive immunoassay. Each sensing zone can correspond to a respective detection channel and a respective target analyte. For example, the sensing zones can be positioned on the nitrocellulose in registration with fluid paths established by the patterned bonding layer so that rehydrated detection reagents encounter the sensing zones during capillary transport. In certain examples, each sensing zone is formed by depositing a solution comprising a capture agent corresponding to the respective target analyte and drying to immobilize the capture agent on the membrane. In some examples, the capture agent can include a target-analyte protein conjugate, and a blocking agent can be applied to reduce non-specific binding. The sensing zones can be printed as transverse lines, dots, or other patterns having predetermined width, length, and spacing suitable for optical readout.

In some examples, component (iii) further includes one or more control regions disposed on the porous membrane. A positive control region can include a capture reagent configured to bind the detection reagents independently of analyte concentration, thereby confirming release of detection reagents and completion of flow. In certain examples, the positive control region is positioned downstream of the sensing zones within one or more detection channels or in a location common to multiple channels. A negative control region can include an area of the membrane that is left untreated or treated only with a blocking agent, thereby providing a baseline for background signal. In various examples, signal characteristics at the control regions can be evaluated to determine whether a given test run is valid.

In an example, the spatial arrangement of the sensing zones and control regions on component (iii) is selected to promote uniform exposure to capillary flow and to minimize cross-talk between channels. For example, the sensing zones corresponding to different target analytes can be separated by predetermined distances along or across the membrane, and channel boundaries can be defined by the patterned bonding layer. In some examples, the sensing zones and control regions are located within an optical access window of the assembled device to facilitate image capture. In certain examples, identifiers, alignment marks, or other visual guides can be printed adjacent to the sensing zones to assist with region localization during optical processing, without limiting the placement or geometry of the underlying capture regions.

In some examples, each sensing zone is formed by depositing a solution of a hormone-protein conjugate onto the nitrocellulose membrane at a defined location and drying to immobilize the conjugate. For cortisol, testosterone, and progesterone detection, the respective capture agents are cortisol-bovine serum albumin (cortisol-BSA), testosterone-bovine serum albumin (testosterone-BSA), and progesterone-bovine serum albumin (progesterone-BSA). Each hormone-BSA conjugate is prepared by covalently linking the hormone to BSA through a carbodiimide or mixed anhydride coupling reaction, producing a multivalent conjugate that presents multiple hormone epitopes on a single protein scaffold. This multivalency increases the density of available binding sites at the sensing zone and promotes efficient competitive capture of labeled antibody conjugates during assay operation.

Deposition of the capture agent onto the nitrocellulose membrane is performed using a contact dispensing or inkjet-style reagent dispenser configured to deliver a defined volume at a defined position with high positional repeatability. In an example, each hormone-BSA conjugate solution is dispensed as a transverse line across the membrane at a concentration of approximately 0.5 to 0.7 mg/mL in phosphate-buffered saline (PBS), and the membrane is dried at approximately 37 °C for approximately one hour to immobilize the conjugate. Although specific conjugate concentrations, deposition methods, blocking agents, and drying conditions are described above, other concentrations, dispensing approaches, blocking chemistries, and conditioning protocols are usable in other examples.

Component (iv) can include a plurality of conjugate pads, positioned laterally with respect to the NC membrane and configured to release detection reagents upon wetting. Each conjugate pad can serve as the conjugate zone of a respective detection channel and can contain detection reagents conjugated to antibodies specific to the respective target analyte of that channel. In some examples, each conjugate pad is positioned to overlap fluidically with the NC membrane so that, upon contact with plasma, the detection reagents are rehydrated and transported by capillary action downstream to the corresponding sensing zone on the porous membrane.

In some examples, the detection reagents carried on each conjugate pad include gold nanoparticles (AuNPs) conjugated to monoclonal antibodies specific to a respective target analyte. The AuNPs serve as visible colorimetric labels whose accumulation at a sensing zone produces a red-to-pink color signal detectable by eye or by a camera. In an example, AuNPs of approximately 20 to 22 nanometers in diameter are synthesized by reduction of gold chloride trihydrate (HAuCl₄) with trisodium citrate, which acts as both a reducing agent and a stabilizing agent. During synthesis, an aqueous HAuCl₄ solution is heated to boiling under continuous stirring, trisodium citrate solution is rapidly injected, and the reaction proceeds for approximately 30 minutes, during which the solution transitions from light yellow through blue to a wine-red color indicative of nanoparticle formation. The resulting AuNP suspension is cooled to room temperature and stored at 4 °C prior to conjugation.

Conjugation of monoclonal antibodies to the AuNP surface is performed by passive adsorption. Before conjugation, the pH of the AuNP suspension is adjusted to approximately 8.0 using potassium carbonate (K₂CO₃) to promote favorable electrostatic interactions between the citrate-stabilized AuNP surface and the antibody. Antibody solution is added to the pH-adjusted AuNP suspension and incubated under moderate agitation for approximately two hours to allow stable adsorption of the antibody layer onto the nanoparticle surface. Following adsorption, bovine serum albumin (BSA) is added as a blocking agent to passivate unoccupied surface sites and reduce non-specific interactions, and the mixture is incubated for an additional 15 minutes. The resulting conjugate suspension is purified by centrifugation to remove unbound antibody and resuspended in a conjugation buffer. In some examples, the conjugation buffer includes BSA, Triton X-100, sucrose, and sodium azide in phosphate-buffered saline (PBS), which together promote conjugate stability, reduce aggregation, and inhibit microbial degradation during storage. For example, the sucrose acts as a cryoprotectant and stabilizer during drying, while the Triton X-100 reduces surface tension to promote uniform pad wetting upon rehydration.

A defined volume of each AuNP-antibody conjugate suspension is deposited onto a respective conjugate pad and dried under controlled conditions. In some examples, the conjugate pads are conditioned at approximately 35 °C and 20% relative humidity for approximately 24 hours in a controlled incubator to achieve uniform drying and stable reagent immobilization. This drying step preserves conjugate activity during storage and ensures consistent rehydration and release upon contact with plasma during assay operation. Although specific synthesis parameters, antibody amounts, blocking agents, and drying conditions are described above, other nanoparticle sizes, conjugation methods, blocking strategies, and conditioning protocols are usable in other examples.

Although gold nanoparticles are described above as the colorimetric label, other label types are usable in other examples, including colored latex particles, fluorescent labels, quantum dots, magnetic particles, and enzymatic labels, provided the selected label generates a detectable signal at the sensing zones under the chosen readout modality.

Component (v) can include a second patterned PSA layer that delineates and seals downstream regions of the device, such as a wicking region, and provides additional bonding and registration for upper layers. In some examples, the geometry of component (v) promotes unidirectional flow from inlet regions across the sensing zones toward the downstream region. Component (vi) can include a wicking pad formed of an absorbent material configured to draw fluid through the device by capillary action. In some examples, the wicking pad geometry is selected to increase effective surface area and promote efficient clearance of residual fluid from the sensing region.

In some examples, the wicking pad of component (vi) has a flower-shaped geometry in which a central body region is surrounded by a plurality of outwardly extending lobes or petals, as shown in the drawing. Each lobe increases the total perimeter and surface area of the wicking pad relative to a rectangular pad of equivalent footprint, and the resulting increase in exposed absorbent area enhances the rate and total capacity of fluid uptake by capillary action. For example, a flower-shaped wicking pad with four to eight lobes can provide a substantially greater effective absorbent perimeter than a rectangular pad occupying the same device footprint, enabling faster fluid clearance from the sensing zones and reducing the risk of backflow or pooling at the downstream end of the detection channels. The lobes are oriented to extend into regions of the device that would otherwise be unoccupied by functional components, making efficient use of available device area without increasing overall device dimensions.

The flower-shaped geometry also promotes uniform fluid distribution across the wicking pad during absorption. As fluid arrives at the wicking pad from multiple detection channels, the lobe geometry distributes the incoming fluid laterally across the pad surface rather than concentrating it at a single point, reducing the risk of localized saturation that could impede continued capillary transport. In some examples, the wicking pad is formed from a high-capacity cellulose or glass fiber absorbent material, and the flower-shaped geometry is defined by laser cutting or die cutting the pad to the desired outline prior to assembly. The wicking pad is positioned within the device such that at least a portion of the pad, including one or more lobes, is exposed to the ambient environment through an opening in the top encapsulation layer, enabling continuous evaporative and absorptive fluid removal throughout the assay. Although a flower-shaped geometry is described above as a preferred form, other area-enhancing geometries, such as star-shaped, comb-shaped, or multi-arm geometries, are usable in other examples to achieve equivalent increases in effective absorbent surface area.

Component (vii) can include a second PET layer serving as a top encapsulation. In some examples, the top encapsulation encloses lateral flow pathways while leaving at least a portion of the wicking region exposed to the ambient environment to enable continuous absorption and reduce backflow. Component (viii) can include a plurality of inlet pads, such as cellulose pads, arranged to receive a whole blood sample. In some examples, each inlet pad is positioned to overlap a corresponding conjugate pad to promote uniform distribution of the sample and consistent initiation of flow across channels.

Component (ix) can include a blood-separation membrane positioned over the inlet pads and configured to selectively retain cellular blood components while permitting plasma to pass into the underlying capillary-based fluidic network. In some examples, component (ix) enables direct use of finger-prick whole blood without external centrifugation or preprocessing. Although specific materials and relative placements are illustrated in FIG. 3B, other materials, thicknesses, patterns, and arrangements can be used in other examples. Although whole blood from a finger prick is described as the primary sample type, other biological matrices are usable in other examples with appropriate adaptation, including serum, plasma, saliva, and urine, provided the sample is compatible with the capillary-driven fluidic network and the competitive immunoassay chemistry employed.

In some examples, the device is fabricated using computer-aided design (CAD) files to define layer geometries, and individual layers are cut from sheet materials using a CO₂ laser cutter. Registration marks on each layer facilitate precise alignment during assembly. Reagent deposition onto the nitrocellulose membrane and conjugate pads is performed using a contact dispensing or inkjet-style system, and assembled devices are conditioned in a controlled-temperature, controlled-humidity incubator prior to packaging.

In some examples, the system further includes sample collection accessories configured to facilitate finger-prick blood collection and transfer to the device. A sterile lancet punctures the fingertip to produce a blood droplet, and a fixed-volume blood collector receives a defined volume of blood from the droplet. A buffer delivery device dispenses a defined volume of assay buffer to each inlet region concurrent with or after application of the sample, promoting conjugate rehydration and consistent capillary flow initiation across all detection channels. Although specific accessory configurations are described above, other collection and transfer tools are usable in other examples.

FIG. 4 depicts time-resolved imaging of capillary flow dynamics in an example multiplex lateral flow immunoassay device according to examples herein. In an example, the imaging is performed by introducing a dyed aqueous solution through the inlet regions of a device as described above with respect to FIGS. 3A and 3B. This dye-based approach renders the advancing fluid front visually distinguishable from the dry membrane, enabling direct observation of flow progression at each time point.

As shown in FIG. 4, a sequence of images captured at successive time points shows capillary-driven flow progressing from the inlet regions through the spatially separated detection channels and across the porous membrane toward the downstream wicking region. Fluid introduced at the inlet regions wets the conjugate pads, enters the porous membrane, and advances uniformly through each detection channel without external pumping or power. In some examples, the fluid front reaches the downstream wicking region within approximately seven to eight minutes of sample introduction.

The time-resolved sequence demonstrates uniform fluid propagation across all detection channels, confirming consistent capillary transport and balanced flow distribution. For example, the dye tracer reveals whether the capillary-driven fluidic network supports stable and symmetric flow without evidence of channel blockage, leakage, or backflow. This balanced flow distribution supports consistent rehydration of detection reagents in the conjugate zones and uniform exposure of the sensing zones to the transported sample, promoting reliable and reproducible competitive immunoassay performance across the plurality of detection channels.

The time-resolved imaging serves as a development tool for evaluating the effect of design parameters, such as channel geometry, membrane selection, and wicking pad configuration, on flow rate, uniformity, and arrival time. These parameters collectively determine whether capillary transport proceeds symmetrically across all channels under a given device configuration. Although specific time points and flow patterns are illustrated in FIG. 4, other flow rates, channel geometries, and arrival times are observable in other examples depending on device configuration, sample composition, and ambient conditions.

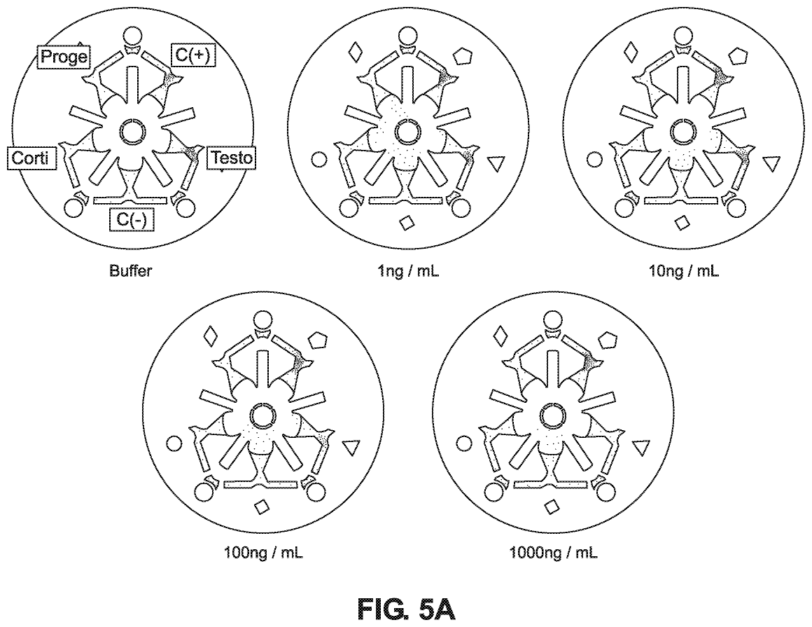

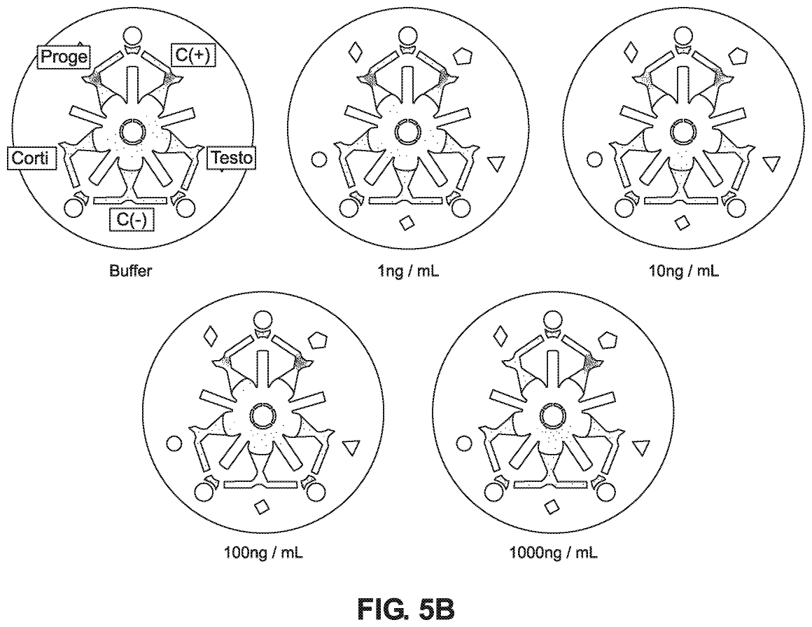

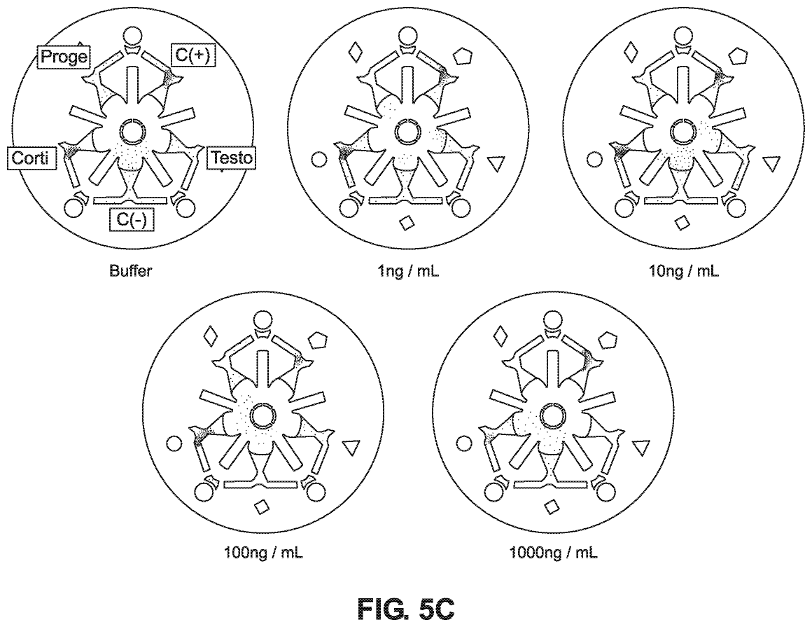

FIGS. 5A-5C depict representative device responses to increasing concentrations of respective target analytes according to examples herein. In the illustrated examples, FIG. 5A shows a series for testosterone, FIG. 5B shows a series for progesterone, and FIG. 5C shows a series for cortisol, each imaged after completion of flow for single-analyte runs spanning a concentration range from 0 to 1000 ng/mL. In some examples, the nitrocellulose membrane is functionalized with a sensing zone corresponding to the respective hormone-protein conjugate and devices are tested at defined serial dilutions alongside a buffer-only condition to establish the full response curve for each analyte.

In an example, within each series of FIGS. 5A-5C, sensing-zone intensity decreases as analyte concentration increases, illustrating a competitive immunoassay response in which signal at the sensing zone varies inversely with analyte concentration. For example, devices run at lower concentrations exhibit stronger sensing-zone signals, and devices run at higher concentrations exhibit progressively weaker signals. Throughout each series, the positive control region remains strong and the negative control region holds at baseline, confirming assay validity across the concentration range.

In some examples, visual aligners adjacent to the regions of interest provide modality-independent identifiers for the channels and controls. The elongated diamond aligner corresponds to progesterone, the circle aligner to cortisol, and the triangle aligner to testosterone. A pentagon aligner identifies the positive control C(+), while an equilateral diamond aligner marks the negative control C(−). These aligners assist with region localization during image capture and processing, maintaining consistent labeling across the concentration series depicted in FIGS. 5A-5C.

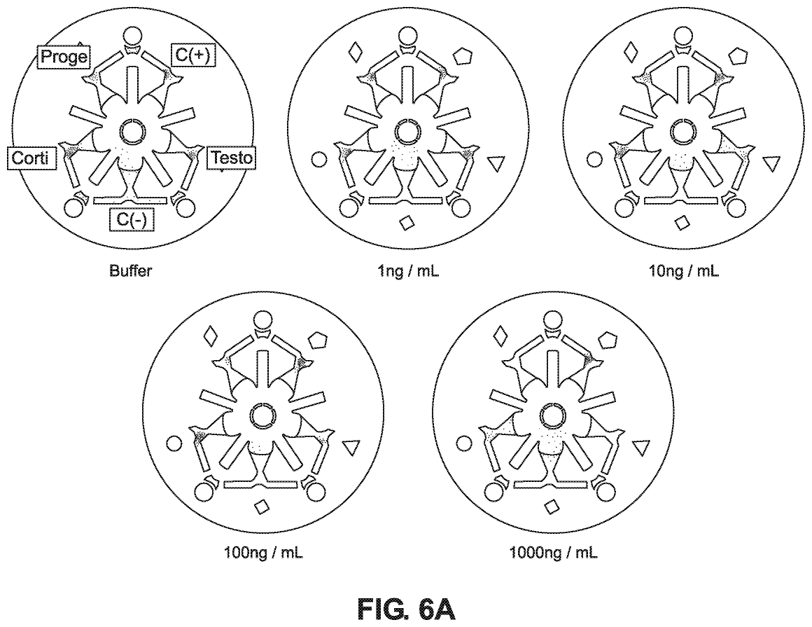

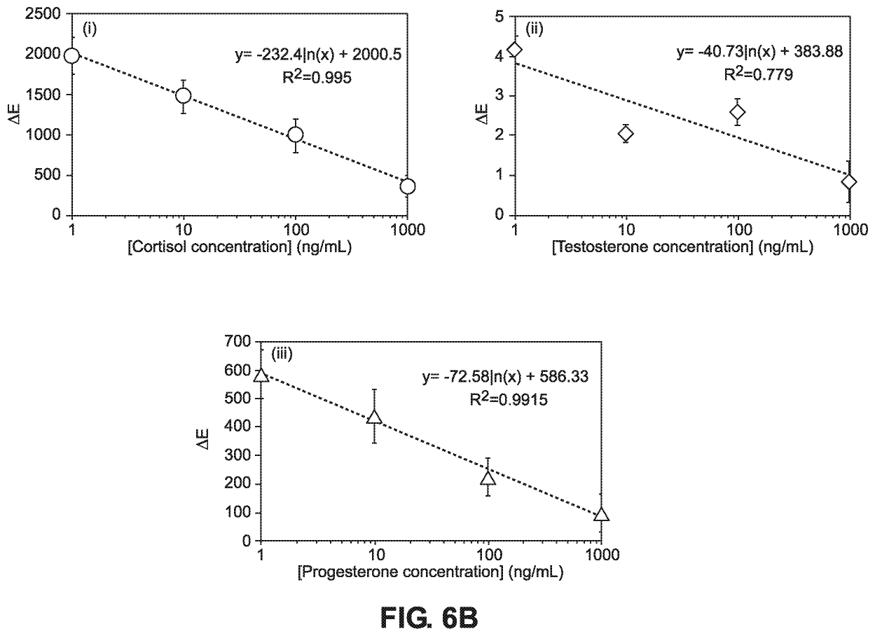

FIGS. 6A and 6B depict representative imaging and calibration data for a multiplex lateral flow immunoassay device according to examples herein. FIG. 6A illustrates example device images acquired after completion of flow for mixed-analyte test runs, while FIG. 6B illustrates example calibration plots mapping an image-derived, color-based metric to analyte concentration for respective target analytes.

In an example, FIG. 6A shows device images captured by a camera, which can be a camera of a mobile device, with the sensing zones visible for respective target analytes. The device includes a calibration region including fiducial markers and color reference standards, and the captured images are normalized using those references prior to quantification. Mixed-analyte runs include combinations of low, medium, and high concentrations for different analytes within a panel, and the sensing-zone intensities reflect the expected inverse response under a competitive immunoassay format. Positive and negative control regions remain within expected ranges in these runs, indicating proper conjugate release and flow completion independent of analyte levels. For example, visual aligners adjacent to regions of interest assist with localization of the respective sensing zones and controls during image processing.

In an example, FIG. 6B shows calibration plots for respective target analytes, where a color-difference metric calculated from processed image data is plotted against known analyte concentration. The color-difference metric is computed relative to a reference and is derived from a perceptually uniform color space, such as CIELAB, which weights color differences in proportion to human visual perception. Calibration data are generated from serial dilutions spanning a defined range and are fit with a model curve suitable for mapping image-derived values to concentrations during subsequent use. Each analyte in the panel has a corresponding calibration curve, and those curves can be validated using images from mixed-analyte runs such as those shown in FIG. 6A. The calibration data are stored in memory of a computing device and accessed by a processor to determine concentrations from captured images of unknown samples.

In some examples, the image processing pipeline described above with respect to FIG. 6B is implemented using a machine learning framework configured to perform segmentation, normalization, and quantitative concentration prediction from smartphone-acquired images of the device. This framework addresses practical challenges of real-world image acquisition, including low-contrast sensing-zone signals at low analyte concentrations, illumination variability across imaging environments, and manufacturing artifacts such as air bubbles and strip imperfections that can interfere with signal extraction.

A first stage of the framework performs sensing-zone segmentation using a modified U-Net architecture enhanced with hybrid channel-spatial attention mechanisms. The U-Net architecture is a convolutional encoder-decoder network that produces pixel-level segmentation maps from input images, and the hybrid attention modules extend this architecture by dynamically weighting both spatial and channel-wise features to emphasize diagnostically relevant regions while suppressing background noise and artifacts. For example, the attention gates selectively amplify signals at sensing zones corresponding to target analytes and control regions while attenuating responses from non-specific regions of the device image. In some examples, the segmentation model is trained using a combination of focal loss and dice coefficient as the composite loss function. Focal loss down-weights the contribution of easy background examples during training, directing learning toward the harder-to-detect low-contrast sensing-zone signals, while the dice coefficient optimizes spatial overlap between predicted and ground-truth segmentation masks. This combination promotes reliable detection of faint sensing-zone signals at low analyte concentrations and robust performance across different device configurations without manual parameter tuning.

Following segmentation, a second stage processes the extracted sensing-zone data using a two-branch neural network. One branch performs intensity normalization across test batches, accounting for run-to-run variability in conjugate release, membrane lot differences, and imaging conditions that would otherwise introduce systematic error into concentration estimates. The other branch analyzes spatial relationships among the plurality of sensing zones, including relative signal intensities across channels and positional relationships among test and control regions, to improve the accuracy and robustness of the quantitative prediction. In an example, outputs from both branches are combined to produce a concentration estimate for each target analyte in the panel. The framework further integrates statistical modeling and uncertainty estimation techniques to assess confidence in each concentration prediction and to identify potential strip defects or assay anomalies that may compromise result validity. For example, a run flagged as having a defect or low-confidence prediction can be indicated to the user through the mobile device interface, prompting a repeat measurement. Although a specific two-stage ML architecture is described above, other segmentation models, normalization strategies, and quantification networks achieving equivalent functions are usable in other examples.

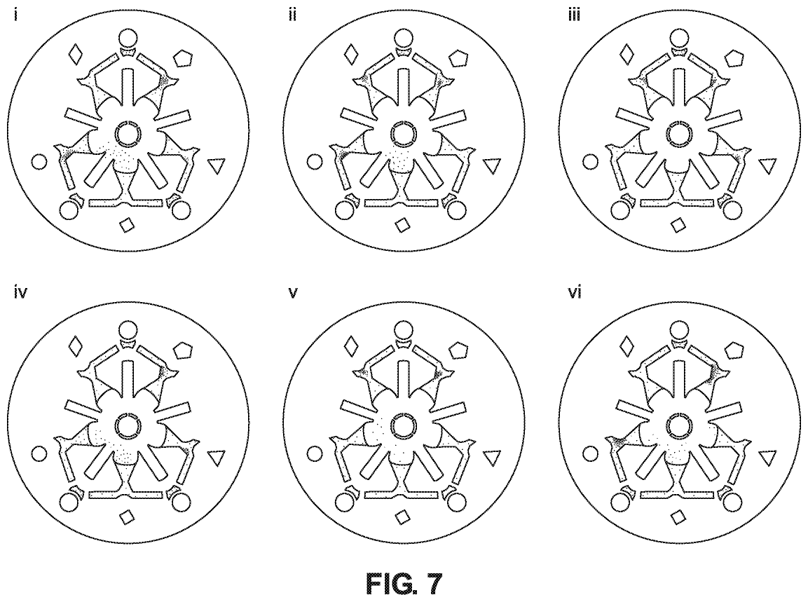

FIG. 7 depicts example selectivity results for a multiplex lateral flow immunoassay device under single-analyte and binary-mixture conditions according to examples herein. These results build on the calibration data of FIG. 6B by demonstrating that each detection channel responds selectively to its respective target analyte even when other analytes are present or absent. In the illustrated example, device images acquired after completion of flow are shown for test runs in which one or two target analytes are present while the remaining analyte is absent.

Single-analyte runs demonstrate that the sensing zone corresponding to the present analyte exhibits a reduced signal consistent with a competitive immunoassay response, while sensing zones corresponding to absent analytes remain at baseline. For example, when only one analyte is introduced, the corresponding sensing zone shows attenuation across the tested concentration level, and the other sensing zones show no appreciable change, indicating a channel-specific response without detectable cross-reactivity.

Binary-mixture runs extend this observation to two simultaneously present analytes. Sensing zones corresponding to both present analytes show reduced signals, while the sensing zone corresponding to the absent analyte holds at baseline. The degree of attenuation at each affected sensing zone is consistent with the concentration of the respective analyte in the mixture, indicating independent behavior of the spatially separated detection channels and confirming that the presence of one analyte does not perturb the response of adjacent channels.

Positive and negative control regions remain within expected ranges across both single-analyte and binary-mixture conditions, confirming conjugate release and flow completion independent of analyte identity. Images captured under standardized conditions are processed to quantify signal levels, and those quantified values corroborate the visual selectivity results by indicating minimal cross-talk among channels under the test conditions.

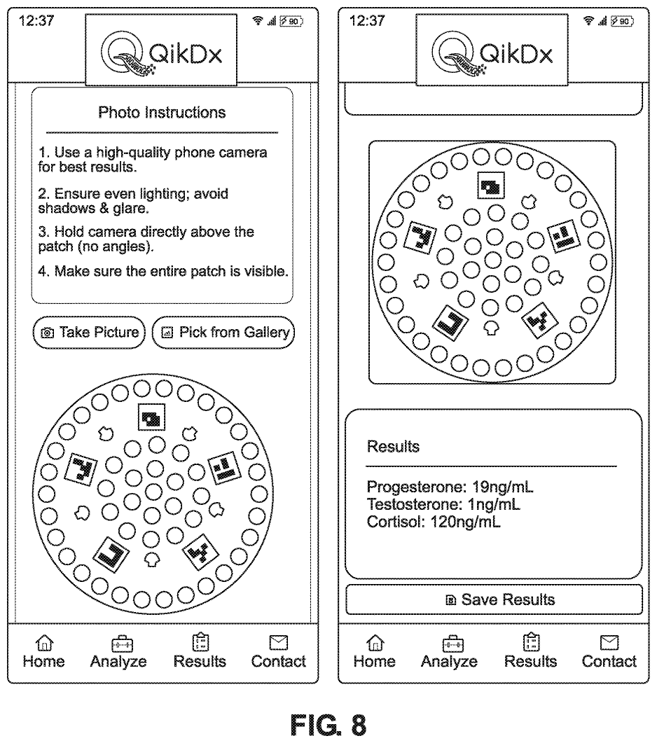

FIG. 8 depicts an example whole-blood operation and mobile device readout for a multiplex lateral flow immunoassay device according to examples herein. Building on the selectivity results of FIG. 7, this figure illustrates end-to-end operation from finger-prick sample application through quantitative concentration output. In the illustrated example, a finger-prick sample is obtained and applied directly to the inlet regions of the device without external preprocessing, and the device operates by capillary action to transport plasma through spatially separated detection channels to respective sensing zones.

In an example, the device includes a blood-separation element disposed at or over the inlet regions and configured to selectively retain cellular blood components while permitting plasma to pass into a capillary-based fluidic network including a porous membrane. After completion of the flow, the sensing zones present visually detectable signals that vary inversely with analyte concentration under a competitive immunoassay format. Positive and negative control regions are evaluated to confirm run validity, consistent with the control behavior described above with respect to FIGS. 5A-5C and FIG. 7.

A camera of a mobile device captures an image of the device after flow completion, and an application executing on the mobile device processes that image to determine analyte concentrations. The device includes a calibration region including fiducial markers and color reference standards, and the application normalizes the captured image using those references before quantifying signal intensity at the sensing zones. This normalization step accounts for illumination variability and camera-to-camera differences, enabling consistent quantitative readout across different mobile devices and imaging environments.

The mobile device user interface presents determined concentrations for respective target analytes alongside indicators of assay validity derived from the control regions. Results are displayable on the mobile device, storable locally, or transmittable to a remote system for further review. Although one workflow and user interface arrangement is illustrated in FIG. 8, other imaging configurations, interface layouts, and output formats are usable in other examples.

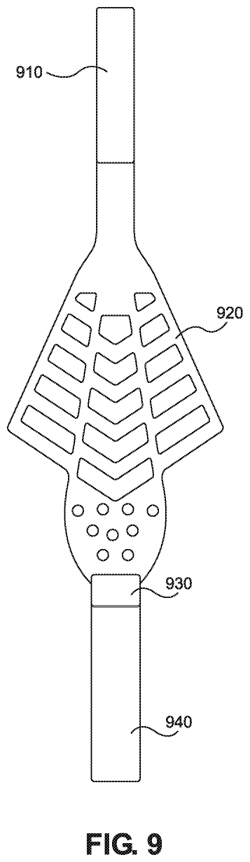

FIG. 9 depicts an example alternative multiplex test strip according to examples herein. Building on the layered device architecture described above with respect to FIGS. 3A and 3B, this example illustrates a branching strip geometry as an alternative configuration for multiplexed lateral flow detection. In this example, the test strip includes a sample pad 940 configured to receive a whole blood sample and to initiate capillary flow through a branching architecture 920 formed on a porous membrane.

In an example, one or more conjugate pads 930 are positioned upstream along respective branches and are configured to release detection reagents upon wetting. Each branch can correspond to a respective detection channel and can include an obliquely oriented sensing region disposed along the branch to facilitate optical access and spatial separation according to examples herein.

The branches 920 extend toward a downstream wicking pad 910 configured to draw fluid through the strip by capillary action. This downstream wicking pad 910 serves the same function as the absorbent region described above with respect to component (vi) of FIG. 3B, maintaining unidirectional flow and promoting clearance of residual fluid from the sensing regions. The branching layout promotes compact placement of multiple channels on a single strip, and the oblique orientation of the sensing regions along the branches assists with channel isolation and image segmentation during optical readout.

In various examples, the sample pad 940 is overlaid by a blood-separation element configured to retain cellular blood components while permitting plasma to pass into the branching architecture 920. The conjugate pads 930 include reagents specific to respective target analytes, and the wicking pad 910 is shaped or sized to support unidirectional flow through the branches. Although specific geometries and component arrangements are illustrated in FIG. 9, other branch counts, sensing region orientations, and pad configurations are usable in other examples.

Other examples of the disclosure will be apparent to those skilled in the art from consideration of the specification and practice of the examples disclosed herein. Though some of the described methods have been presented as a series of steps, it should be appreciated that one or more steps can occur simultaneously, in an overlapping fashion, or in a different order. The order of steps presented is only illustrative of the possibilities, and those steps can be executed or performed in any suitable fashion. Moreover, the various features of the examples described here are not mutually exclusive. Rather any feature of any example described here can be incorporated into any other suitable example. It is intended that the specification and examples be considered as exemplary only, with a true scope and spirit of the disclosure being indicated by the following claims.

What is claimed is:

1. A multiplex lateral flow immunoassay device for the detection of a plurality of analytes in a whole blood sample, the device comprising:
a capillary-driven fluidic network defined by at least a substrate layer, one or more bonding layers patterned to form a plurality of microfluidic channels, and a porous membrane disposed within the channels, the fluidic network extending from a plurality of inlet regions through the porous membrane;
a blood-separation element disposed at or upstream of each inlet region, the blood-separation element configured to selectively retain cellular blood components while permitting plasma to pass into the fluidic network;
a plurality of spatially separated detection channels formed within the fluidic network, each detection channel comprising:
an inlet region,
a conjugate zone containing detection reagents conjugated to antibodies specific to a respective target analyte, and
a sensing zone on the porous membrane coated with a capture agent corresponding to the respective target analyte,
wherein the device is configured to perform a competitive immunoassay in each detection channel such that signal intensity at each sensing zone is inversely correlated with the concentration of the respective target analyte in the sample.

2. The device of claim 1, wherein the porous membrane comprises nitrocellulose and the one or more bonding layers comprise a pressure-sensitive adhesive.

3. The device of claim 1, wherein each detection channel is fluidically isolated from the other detection channels.

4. The device of claim 1, wherein the detection reagents comprise gold nanoparticles conjugated to monoclonal antibodies.

5. The device of claim 4, wherein the gold nanoparticles have a mean diameter between about 15 nm and about 30 nm.

6. The device of claim 1, wherein the blood-separation element comprises a plasma separation membrane configured to retain red blood cells while permitting plasma to pass.

7. The device of claim 1, further comprising a calibration region on the device including fiducial markers and color reference standards to facilitate optical readout.

8. A method for detecting a plurality of analytes in a whole blood sample using a multiplex lateral flow immunoassay device, the method comprising:
applying the whole blood sample to a plurality of inlet regions of the device, the device having a blood-separation element, a capillary-driven fluidic network including a porous membrane, and a plurality of spatially separated detection channels;
separating cellular components from the sample with the blood-separation element to yield plasma that enters the fluidic network;
transporting, by capillary action, the plasma and detection reagents in each detection channel through the porous membrane to a respective sensing zone corresponding to a respective target analyte;
inducing competitive binding such that a signal intensity at each sensing zone varies inversely with concentration of the respective target analyte; and
acquiring an optical measurement of the sensing zones and determining concentrations of the plurality of analytes based on stored calibration data.

9. The method of claim 8, wherein acquiring the optical measurement comprises capturing an image with a camera of a mobile device and analyzing the image with software executing on the mobile device.

10. The method of claim 8, wherein determining concentrations comprises converting image data to a perceptually uniform color space and calculating a color-difference metric relative to a reference standard.

11. The method of claim 8, further comprising normalizing the optical measurement using fiducial markers and color reference standards on the device.

12. The method of claim 8, wherein the detection reagents in each detection channel comprise gold nanoparticles conjugated to monoclonal antibodies specific to the respective target analyte.

13. The method of claim 8, wherein the whole blood sample is a finger-prick sample and the separating step is performed by a plasma separation membrane that retains red blood cells.

14. A system for detecting a plurality of analytes in a whole blood sample, the system comprising:
a multiplex lateral flow immunoassay device having:
a blood-separation element;
a capillary-driven fluidic network including a porous membrane; and
a plurality of spatially separated detection channels formed within the fluidic network, each with a respective sensing zone,
wherein the device is configured to perform a competitive immunoassay in each detection channel such that signal intensity at each sensing zone varies inversely with concentration of a respective target analyte;
a camera configured to capture an image of the sensing zones;
a processor;
a memory storing calibration data and instructions that, when executed by the processor, cause the processor to:
quantify signal intensity at each sensing zone relative to a reference;
determine concentrations of the plurality of analytes based on the calibration data; and
generate an output indicating the concentrations.

15. The system of claim 14, wherein the camera, memory, and processor are implemented in a single mobile device.

16. The system of claim 14, wherein the device includes a calibration region with fiducial markers and color reference standards, and the instructions cause the processor to normalize the image using the fiducial markers and the color reference standards.

17. The system of claim 14, wherein each detection channel is fluidically isolated from the other detection channels.

18. The system of claim 14, wherein the device includes detection reagents comprising gold nanoparticles conjugated to monoclonal antibodies specific to the respective target analyte.

19. The system of claim 14, wherein the device further comprises one or more bonding layers patterned to form a plurality of microfluidic channels.

20. The system of claim 14, wherein the bonding layers comprise a pressure-sensitive adhesive.
